(19) United States
(12) Patent Application Publication
(10) Pub. No.: US 2021/0174954 A1
(43) Pub. Date: Jun. 10, 2021

(54) MONITORING A PATIENT WITH CHRONIC OBSTRUCTIVE PULMONARY DISEASE

(71) Applicant: KONINKLIJKE PHILIPS N.V., Eindhoven (NL)

(72) Inventors: WILLIAM TRUSCHEL, MONROEVILLE, PA (US); FRANCESCO VICARIO, BOSTON, MA (US); MICHAEL POLKEY, MONROEVILLE, PA (US); PABLO Andres Nanez Ojeda, MONROEVILLE, PA (US)

(21) Appl. No.: 17/094,022

(22) Filed: Nov. 10, 2020

Related U.S. Application Data
(60) Provisional application No. 62/943,573, filed on Dec. 4, 2019.

(51) Int. Cl. G16H 40/67; G16H 10/60; G16H 50/30; G16H 50/70; G16H 40/20; A61B 5/11; A61B 5/01; A61B 5/00

(52) U.S. Cl. CPC G16H 40/67; G16H 10/60; G16H 50/30; G16H 50/70; G16H 40/20; G16H 50/50; A61B 5/015; A61B 5/7275; A61B 5/7475; A61B 5/0008; A61B 5/1113

(57) ABSTRACT

A system and method for determining a patient condition includes receiving sensor information over an observation period, generating one or more metrics based on the sensor information, comparing the one or metrics to reference information to determine deviation information, and generating a risk score indicating a probability that a patient is experiencing an COPD-related event based on the deviation information. The sensor information is received from one or more sensors at predetermined locations of a patient location.

### CROSS-REFERENCE TO RELATED APPLICATIONS

This patent application claims the priority benefit under 35 U.S.C. § 119(e) of U.S. Provisional Application No. 62/943,573 filed on Dec. 4, 2019, the contents of which are herein incorporated by reference.

### BACKGROUND OF THE INVENTION

### 1. Field of the Invention

The present invention pertains to a system and method for processing information to determine the condition of a patient.

### 2. Description of the Related Art

Patient monitoring has been performed by various rudimentary applications. For example, some patients are given medallions which can be used to contact help if they fall or otherwise become immobilized. Other patients are given recording devices that monitor heart rhythm for use in diagnosing heart disease. In other cases, patients are monitored at a sleep facility for the purpose of diagnosing sleep apnea. All of these approaches have proven to be inconvenient, disruptive, invasive, or otherwise require significant participation on the part of the patient. Also, none of these approaches apply to predicting or monitoring the progression of diseases, including but not limited to breathing-related diseases such as chronic obstructive pulmonary disease (COPD).

### SUMMARY OF THE INVENTION

Accordingly, it is an object of the present invention to provide a system and method for monitoring a patient condition that is not disruptive or invasive and is convenient for the patient. It is another object of the invention apply provide such a system and method to predict and monitor the onset and progression of diseases, including but not limited to breathing-related diseases such as COPD.

These and other objects of the disclosed embodiments are achieved by providing a method for determining a patient condition which includes receiving sensor information over an observation period, generating one or more metrics based on the sensor information, comparing the one or metrics to reference information to determine deviation information, and generating a risk score indicating a probability that a patient is experiencing an COPD-related event based on the deviation information, wherein the sensor information is received from one or more sensors at predetermined locations of a patient location. The one or more sensors may include one or more first sensors to track movement of the patient at the patient location throughout the observation period. The one or more first sensors may track three-dimensional movement of the patient and the three-dimensional movement may include movement up or down stairs.

The one or more metrics may include a heat map generated based on the information received from the one or more first sensors. Generating the one or more metrics may include generating the heat map to include a first coverage area including one or more routes of movement taken by the patient at the patient location during the observation period. Comparing the one or more metrics to reference information may include comparing the first coverage area to second coverage area of a heat map corresponding to reference information, the second coverage area corresponding to movement at the patient location for a non-COPD-related event and the deviation information corresponding to a difference between the first coverage area and the second coverage area.

The one or more sensors may include one or more second sensors to generate physiological data for the patient. Comparing the one or metrics to reference information may include comparing a first metric generated based on the movement tracking information received from the one or more first sensors to first reference information to generate a first deviation value, and comparing a second metric generated based on the physiological data to second reference information to generate a second deviation value, and wherein the risk score is generated based on the first deviation value and the second deviation value.

In accordance with one or more other embodiments, a monitoring system, comprising an interface to receive sensor information over an observation period, a memory storing instructions for determining a health condition of a patient, and a processor to execute the instructions to perform operations including: generating one or more metrics based on the sensor information, comparing the one or metrics to reference information to determine deviation information, and generating a risk score indicating a probability that a patient is experiencing an COPD-related event based on the deviation information, wherein the sensor information is received from one or more sensors at predetermined locations of a patient location. The one or more sensors include one or more first sensors to track movement of the patient at the patient location throughout the observation period. The one or more first sensors may track three-dimensional movement of the patient and the three-dimensional movement may include movement of the patient up and down stairs. The one or more metrics may include a heat map generated based on the information received from the one or more first sensors.

The processor may generate the one or more metrics by generating the heat map to include a first coverage area including one or more routes of movement taken by the patient at the patient location during the observation period. The processor may compare the one or more metrics to reference information by comparing the first coverage area to a second coverage area of a heat map corresponding to reference information, the second coverage area corresponding to movement at the patient location for a non-COPD-related event and the deviation information corresponding to a difference between the first coverage area and the second coverage area. The one or more sensors include one or more second sensors to generate physiological data for the patient.

The processor may compare the one or metrics to reference information by operations including: comparing a first metric generated based on the movement tracking information received from the one or more first sensors to first reference information to generate a first deviation value, and comparing a second metric generated based on the physiological data to second reference information to generate a second deviation value, and wherein the risk score is generated based on the first deviation value and the second deviation value.

In accordance with one or more embodiments, a monitoring system includes interface means for receiving sensor information over an observation period, a memory storing instructions for determining a health condition of a patient, and processing means for performing operations including: generating one or more metrics based on the sensor information, comparing the one or metrics to reference information to determine deviation information and generating a risk score indicating a probability that a patient is experiencing an COPD-related event based on the deviation information, wherein the sensor information is received from one or more sensors at predetermined locations of a patient location. The one or more sensors may include one or more first sensors to track movement of the patient at the patient location throughout the observation period. The one or more metrics include a heat map generated based on the information received from the one or more first sensors.

The processing means may perform comparing the one or more metrics to reference information by: generating a first coverage area of the heat map corresponding to routes taken by the patient at the patient location during the observation period, the coverage area and routes generated based on the information received from the one or more first sensors; and comparing the first coverage area to a second coverage area in a heat map corresponding to reference routes of movement at the patient location for a non-COPD-related event, wherein the deviation information corresponds to a difference between the first coverage area and the second coverage area.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a view illustrating an embodiment for monitoring a patient;

FIG. 2 is a view illustrating an embodiment of a monitoring system;

FIG. 3 is a view illustrating an embodiment of a method for monitoring a patient;

FIG. 4 is a view illustrating an example of detecting a COPD-related event based on one type of deviation information;

FIG. 5 is a view illustrating an embodiment of routing information which may be used as a basis for detecting a COPD-related event;

FIG. 6A is a view illustrating examples of a reference heat map, FIG. 6B is a view illustrating a heat map corresponding to a first risk level for a COPD-related event, and FIG. 6C is a view illustrating a second risk level for a COPD-related event;

FIG. 7 is a view illustrating an additional example of how metric and deviation information may be generated for purposes of detecting a COPD-related event;

FIG. 8 is a view illustrating an example of a gait pattern of a patient;

FIGS. 9A to 9E are views of graphs of data that correspond or may be used as a basis for generating metrics indicative of a COPD-related event;

FIGS. 10A to 10C are views of graphs of data that correspond or may be used as a basis for generating additional metrics indicative of a COPD-related event;

FIG. 11 is a view illustrating an example of a set of stairs with a relative three-dimensional data axis;

FIGS. 12A and 12B are views graphs of data that correspond or may be used as a basis for generating additional metrics indicative of a COPD-related event;

FIG. 13 is a view illustrating an example of data that correspond or may be used as a basis for generating additional metrics indicative of a COPD-related event;

FIG. 14 is a view illustrating an example of data that correspond or may be used as a basis for generating additional metrics indicative of a COPD-related event;

FIG. 15 is a view illustrating an example of data that correspond or may be used as a basis for generating additional metrics indicative of a COPD-related event;

FIG. 16 is a view illustrating an example of data that correspond or may be used as a basis for generating additional metrics indicative of a COPD-related event;

FIG. 17 is a view illustrating an example of data that correspond or may be used as a basis for generating additional metrics indicative of a COPD-related event;

FIG. 18 is a view illustrating an example of data that correspond or may be used as a basis for generating additional metrics indicative of a COPD-related event; and

FIG. 19 is a view illustrating an example of data that correspond or may be used as a basis for generating additional metrics indicative of a COPD-related event.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

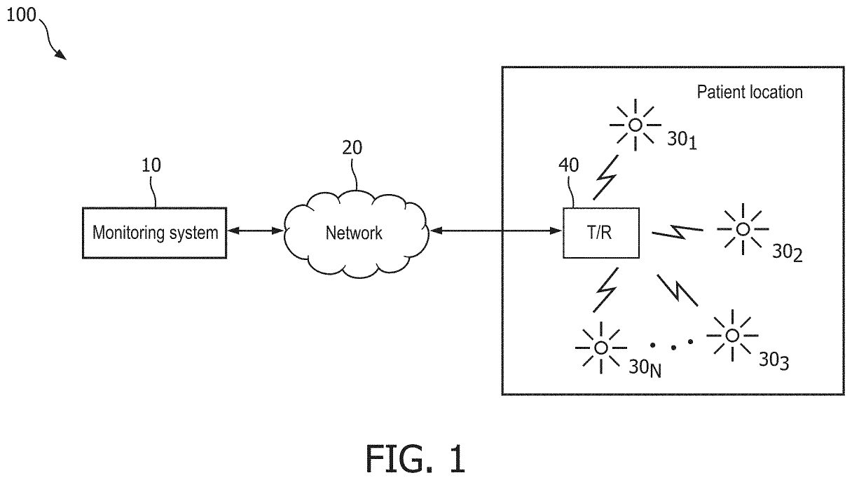

FIG. 1 schematically illustrates an exemplary embodiment of a system 100 for monitoring a patient for the purpose of performing early warning detection of a disease (and/or detecting the progression of a known disease). The disease may be, for example, any disease that is detectable based on information derived from sensor information. In one embodiment (discussed in greater detail below), the disease is chronic obstructive pulmonary disease detected based on activity or behavioral patterns, taken alone or in combination with vital signs or other physiological data of a patient. The disease may be a different disease detected or predicted, for example, based on different sensor data in another embodiment.

Referring to FIG. 1, the system 100 includes a monitoring system 10 connected to one or more sensors 30₁ to 30_{N} through one or more communications networks 20. The one or more sensors may be arranged at predetermined positions at patient location 5. The sensors may all be of the same type or may be different types. The type(s) of sensors may be determined, for example, based on the specific kind of activity and/or behavioral patterns or physiological data to be detected for the patient.

In a COPD application, a first set of sensors 30₁ to 30_{N} may be, for example, motion detection, position, location, tracking, or other forms of activity sensors arranged at strategic positions throughout the patient location. A second set of sensors 30₁ to 30_{N} may be on the body of the patient. For example, the second set of sensors may include wrist or arm motion sensors for detecting corresponding arm and/or hand motion patterns. A third set of sensors may include leg, angle, and/or foot sensors for detecting motion patterns for corresponding portions of the body of the patient. A fourth set of sensors may include activity sensors different from motion detection sensors. In one embodiment, only one sensor may be included.

The system may include a base station 40 for communicating with the one or more sensors 30₁ to 30_{N}. In one embodiment, base station 40 is connected to the one or more sensors through a wireless protocol, such as, but not limited to, a Wi-Fi or Bluetooth protocol. In another embodiment, a wired connection may be used to connect one or more of the sensors to base station 40. In yet another embodiment, a combination of wired and wireless connections may be used to establish communications between base station 40 and the sensors.

The patient location may be a medical facility, home, residence, or other location of the patient. When in the home or residence, system 100 is especially convenient and beneficial for purposes of obtaining data that captures the everyday life of the patient, where disease is most likely to manifest itself under un-stressful or realistic conditions. In one implementation, one or more sensors 30₁ to 30_{N} may be arranged at strategic locations in the home that have the best chance of capturing patient activity or behavior that may manifest as symptomatic of a disease. These locations include, for example, stairs, bedroom, bathroom, hallway, stairs or steps, or one or more other locations. Additional examples are discussed in greater detail below.

In operation, base station 40 receives signals from the sensors indicative of motion patterns, activity patterns, behavioral patterns, physiological data, and/or other patient-related characteristics. Information indicative of these patterns and characteristics may be processed to generate metrics that may be used as a basis for detecting, predicting, and/or otherwise determining the condition of the patient relative to a particular type of disease or other condition. In one embodiment, base station 40 may pre-process the information obtained from the sensors and then transmit the processed information to monitoring system 10. In another embodiment, base station 40 may transmit the information in raw form to the monitoring system. In another embodiment, the monitoring system itself may be included in or correspond to base station 40.

Once the patient condition is determined, a notification including information indicative of the results may be transmitted to predetermined parties, systems, or devices. In one embodiment, the notification may be transmitted to a responsible party such as a family member, guardian, emergency service, or healthcare professional for the purpose of receiving and/or making care decisions. The patient may also be notified at this time. The notification may be, for example, by email, text message, display, warning, or other forms of transmitted information.

In one embodiment, the one or more sensors may receive information from base station 40 (and/or the monitoring station when separate from the base station). The information may include signals, for example, to control the sensors to operate in certain modes or at certain times. The information may also synchronize operation of the sensors and/or ping the sensors for the purposes of receiving information regarding the patient. In one embodiment, one or more of the sensors may communicate with one or more of the other sensors to control activation and/or operation or synchronization.

Communications network 20 may be the internet, a virtual private network, a mobile communications network, and/or any one of a number of other types of networks that are suitable for communicating data from the base station to monitoring system 10. In this case, base station 40 may include, or be coupled to, a network server. When the base station is not included at the patient location, the sensors may themselves communicate with the monitoring system directly through the network.

Monitoring system 10 may process the information derived and transmitted from the sensors in accordance with the embodiments described herein. In order to receive the sensor information, monitoring system 10 may include or be coupled to a network server.

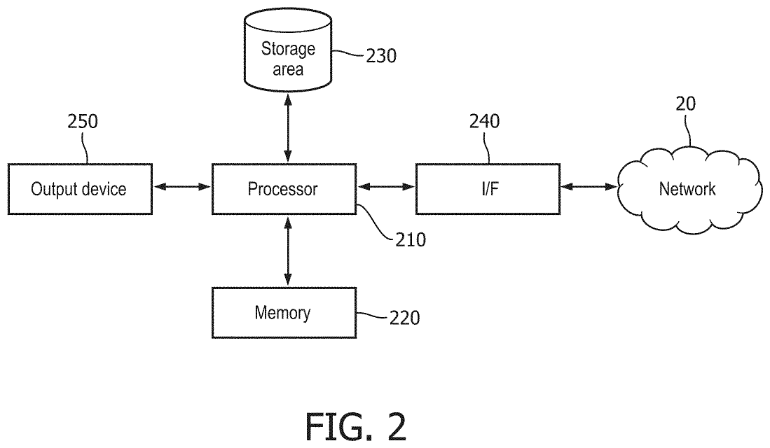

FIG. 2 illustrates an embodiment of monitoring system 10 in FIG. 1. In this embodiment, the monitoring system includes a processor 210, a memory 220, a storage area 230, and interface 240, and an output device 250. Processor 210 may process the patient information received from base station 40 (and/or directly from one or more of sensors 30₁ to 30_{N}) through communications interface 240. The information may be received through network 20 and may be used as a basis for detecting or predicting the presence of a disease, the progression of a known disease, and/or an episode the patient might be experiencing as a result of the disease or otherwise. Processor 210 may include logic implemented in hardware, software, or both, for implementing the operations described herein.

Memory 220 may store instructions including one or more algorithms for controlling processor 210. The algorithms may implement, for example, one or more machine-learning models, neural networks, or other computational engines for performing the analyses of the monitoring system. The machine-learning models may be based on linear regression, reinforced learning techniques, and/or other model-based approaches. The memory may be any one of a variety of a non-transitory computer-readable mediums for storing the instructions for controlling the processor.

Storage area 230 may be one or more databases or other storage devices that may store the data received from the base station and/or sensors. Also, the storage area may store results of the processing performed by the processor, as well as any training data that may be used to implement the model(s) or algorithms used to analyze the sensor data and, ultimately, to detect or predict the presence of a disease, progression of a known disease, and/or an episode a patient might be experiencing as a result of the disease or otherwise.

Output device 250 may be, for example, a display device coupled to the monitoring system. The output device may display the results of the analysis performed by the processor in various ways, including text, graphics, statistical presentations, video, animation, images, and/or other information. The display device may be at the same location as processor 210 or may be remotely located from the processor, for example, to allow for information sharing among health care professionals and/or other concerned parties.

The embodiments discussed below pertain to COPD embodiments; however, the monitoring system may process information in association with other types of diseases in other embodiments.

### COPD Implementation

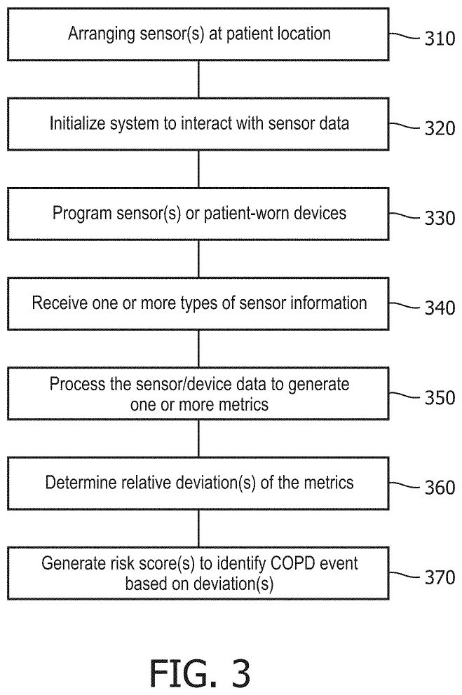

FIG. 3 illustrates an embodiment of a method for monitoring a patient for the purpose of performing early warning detection or prediction of COPD, the progression of COPD, and/or episodes relating to this type of disease. The method may be performed by the monitoring system illustrated in FIGS. 1 and 2 or by another system.

Referring to FIG. 3, the method includes, at 310, arranging one or more of sensors 30₁ to 30_{N} at predetermined positions at a patient location, which for illustrative purposes will be assumed to be the house of a patient to be monitored. The sensors may include one or more beacons, cameras, thermal imaging devices, motion detection sensors, proximity sensors, tracking sensors, GPS sensors, and/or other types of sensors for detecting movement, activity, or other patterns of the patent, as well as patient-worn sensors for acquiring physiological data.

At 320, information of the patient location is stored for access by base station 40 in the home that is to interact with the sensor(s). The information may include two- and/or three-dimensional maps of the patient house, which has been pre-stored in digital form into the processing logic of the base station and/or monitoring system remotely coupled to the base station. This location information may be obtained, for example, based on house blueprints and/or may be generated by imaging technology, e.g., placing cameras throughout the house that take 360° images or video of each room in the house. The two-dimensional map may be, for example, a floor plan of each room and floor of the house of the patient. Examples of the rooms include the kitchen, bathrooms, living areas and garage, as well as other rooms. The map may also include various house features including, but not limited to, doors, appliance locations, fireplace, dinner table, couches and office areas, as well as other features. A three-dimensional map may include, for example, various types of transition areas between the floors such as steps, stairways, ramps, and/or other three-dimensional house features where the patient may walk or otherwise occupy. The sensors may be strategically located throughout all or selected areas in the interior and/or exterior locations of the house for purposes of monitoring activity patterns and other patient-related behavior. In one embodiment, a virtual reality system may be used to generate the map(s).

In addition to sensors the 30₁ to 30_{N}, the base station 40 may communicate with one or more sensors or devices worn on the patient as previously mentioned. These sensors or devices may include vital sign sensors that measure various patient parameters and may include heart rate monitor, blood pressure monitor, oximeter, respiratory rate monitor, etc. In one embodiment, the sensors may also include other types that capture physiological data that may be used as a basis, for example, to detect a COPD-related event.

At 330, sensor(s) 30₁ to 30_{N} and/or patient-worn devices are programmed or otherwise controlled to operate at particular times or according to predetermined modes or schedules. This may be performed based on instructions stored in the monitoring system (e.g., base station 40 or monitoring system 10 operating in communication with the base station and/or directly with the sensor(s)) and/or various programmable modes of the sensors themselves. The sensors and patient-worn devices may be coupled to base station 40, for example, through a short-range connection (e.g., Bluetooth) or by a Wi-Fi connection. In one embodiment, base station 40 may include or be coupled to a router and modem for communicating with the monitoring system through a network. In another embodiment, the sensor(s) and/or patient-worn devices may wirelessly communicate with the monitoring device through the network, without going through a router or the base station. In another embodiment, the monitoring system may itself be included in the patient home, e.g., operating in the role of the base station. For purposes of illustration, the base station and monitoring system 10 will be synonymously referred to as the monitoring system.

At 340, the monitoring system begins receiving information from one or more sensors) 30₁ to 30_{N}, which may or may not include the patient-worn devices. The information may include one type of information (e.g., information from one type of sensor) or a plurality of types of information (e.g., information from multiple types of sensors) over an observation period. For example, the received information may include only tracking or location information generated from the IPS. In another embodiment, tracking or location information may be received simultaneously with patient-worn sensor data. This information may then be stored for processing to detect or predict a COPD-related event.

At 350, in addition to storing this information (e.g., in storage area 230), processor 210 of the monitoring system may process the received information based on the one or more algorithms stored in memory 220. This may involve processing the sensor, physiological, and/or other received information to compute, determine, or otherwise observe one or more predetermined metrics that provide a basis for the detection or prediction of COPD, the progression of the COPD disease (e.g., to a worse or chronic state), and/or an episode that might be expected to occur in a patient as a result of COPD.

The computed metrics may include, for example, arm movement, body posture, position, orientation, and gait speed of the patient. The arm movement may be determined, for example, based on imaging information captures by one of the sensors and/or based on a motion sensor (e.g., incorporated within a watch-like device) worn by the patient. The position of the patient (e.g., standing position, sitting position, etc.) or posture of the patient (e.g., bent over, slumped, slouching, etc.) may be detected by many of the same sensors. The speed of movement of the patient may be determined, for example, by an accelerometer worn by the patient, a tracking system which tracks the movement of the patient as he walks, an image processing system that identifies and follows movement of the patient based on image information, and/or data from other types of sensors.

In addition to these metrics, other types of activity and behavior patterns corresponding to the following metrics may be determined based on processing the sensor/device information: the speed of sitting up, the speed of going from a sitting position to a standing position, the speed of ascending or climbing steps or stairs, walking or stepping patterns, whether the patient has fallen (e.g., stumbled, passed out, fainted, etc.), and whether the patient is using the handrail to ascend or descend the stairs.

Additional metrics include heat maps and patterns obtained by the thermal imaging sensors, beacons, anchors, or other tracking devices to determine, for example, how long the patient is located in each room throughout the day, the rooms visited by the patient throughout the day, and movement patterns of the patient throughout the house. The movement patterns may include, for example, routes taken from one room to another (as determined, for example, by beacon signals, thermal imaging, camera imagery, and/or other types of sensor data), paths commonly traversed by the patient (e.g., trips to the bathroom, trips to the refrigerator, number of times going outside, etc.), and paths not commonly traversed by the patient at all or at least in consideration of the time of day.

In one embodiment, the number of trips the patient makes to the bathroom over a predetermined period of time (e.g., entire day, part of a day, certain hours, etc.) may be counted as a metric. Also, the number of trips the patient makes to the refrigerator may also be a metric. These and other metrics mentioned herein may correlate to certain comorbidities that are sensitive to diet. These other metrics may include, but are not limited to, time out of bed. For example, COPD patients tend to wake up earlier when symptoms leave them restless. Accordingly, the time a patient gets out of bed may constitute a metric that is recorded. Another metric may include time of day. For example, when COPD symptoms occur in the morning, varieties of movement in the morning in the morning may be compared to movement varieties during other times of the day. Thus, another metric may include categorizing movement at different periods of the day, e.g., a morning period and a non-morning period. If movement is restricted in the morning before medications are taken, then the indication of worsening may be assumed. Another metric may include time in the shower, when, for example, a sensor tag is waterproof. Another metric may include times when the patient is detected to be sedentary. For example, the times a patient is sedentary may be expressed as a percentage daily, e.g., sedentary (limited to no movement) for 80% of the day. In one embodiment, these and other metrics described herein may be generated and recorded over time to allow for various trends to be recognized for purposes of COPD disease detection or prediction, and/or for determining the progression of such a disease in the patient.

At 360, the processor of the monitoring system implements one or more algorithms to determine relative deviations in one or more of the metrics. This may involve comparing metrics generated from current sensor or device data to past metrics, which, for example, have been established over time as corresponding to a reference baseline, e.g., what is considered for this particular patient to be “normal” behavior or activity patterns. When the deviation for a given metric (or combination of metrics determined by machine-learning or other experiential evidence) exceeds a predetermined relative threshold amount (e.g., Δ=10% or more), then the processor of the monitoring system may generate a signal indicating that the current metric(s) may be indicative of COPD or that the COPD condition has worsened (e.g., entered a chronic state) or that an episode has occurred as a result of a COPD-related incident.

In some implementations, all of the metrics may be evaluated against disease progression, for example, by tracking the metric daily and detecting a downward spiral, an outlier, a steady change or an alarming pattern that is outside the norm for the specific patient or a population of patients with COPD. Using an objective tracking system may be more reliable than surveying and self-reporting in some circumstances because it eliminates recall bias. (Studies that have surveyed patients and require self-reporting about their symptoms and their relation to physical activity have often failed due to the low patient compliance or participation or inaccurate reporting.)

At 370, the severity and nature of the deviation may be determined to be sufficient to draw such conclusions based on how the model(s) used by the processor of the monitoring system have been trained. For example, in one embodiment the processor may generate one or more risk scores indicative of a probability that the patient is experiencing a COPD-related event. The value of the score may indicate the severity or degree of likelihood of the COPD-related event.

Once the model executed by the processor returns a result indicating that a COPD-related event has occurred based on the relative deviation of one or more of the metrics, the processor may generate information indicating the same for output to the output device (e.g., display device 250) in order to alert or otherwise notify healthcare providers or one or more other responsible parties of the event. The healthcare providers may then respond with help.

For example, if the deviation falls within a first range (e.g., a moderate range), an email, text, or other message may be automatically generated and sent to the patient indicating that he should make an appointment concerning a possible COPD condition. If the deviation falls within a second range (e.g., greater than the first range), then a live telephone call or other urgent message may be made to notify the patient. If the deviation falls within a third range (e.g., greater than the second range), emergency personnel may be contacted to immediately go to the patient location to provide care. The method of notification may be different in other embodiments. Examples of these and other types of deviations are described in greater detail below.

The following provides more detailed and/or additional examples of sensor and other types of systems may be used to generate information for obtaining metrics for a COPD implementation of the system and method. The first system may be an indoor positioning system (IPS) placed in the home of the patient. The indoor positioning system may include beacons, motion detectors, and/or other types of devices or systems that may be used to determine the position of the patient in the home at any given time. In one embodiment, the indoor positioning system may include a real-time location system (RTLS) that transmits and/or receives ultra-wide band (UWB) radio signals to/from the monitoring system, either directly or through a base station, server, or other network-attached device.

A routing system may be used to determine common (e.g., daily) pathways that are traversed by the patient over a period of time, e.g., daily. This system may be implemented, for example, based on the position signals generated by the indoor positioning system or may include other types of sensors.

A usage tracking system may be used to track the location of the patient over each day and compute the percentage of occupied space over the day in relation to the coverage area of the IPS. This may be performed, for example, based on the heat map of the patient location over the coverage area, as previously described.

A system that measures the gait speed of the patient may track with suitable bandwidth the time-ordered coordinates of the location of the patient as he moves along commonly traversed pathways. The change in coordinates may be measured over time in order to compute the gait speed. In one embodiment, the coefficient of variability in pedometer measurements of the patient may be used as a basis for computing gait speed.

A system which interprets changes in information indicative of patient movement and three-dimensional (3D) patient location over time to determine stairway usage of the patient, the speed at which steps are traversed on the stairway, whether or not the patient uses the handle while going up or down the stairs, and the path taking while traversing the stairs, as well as other information. Such a system may be implemented, for example, by a reinforced learning model that is trained based on data indicating altitude and other patient-specific information occurring on a day-to-day basis. After the model is changed, the altitude (3D) information changes, patient location and tracking, and other sensor information may then be linked to stairway usage and its associated features.

Additional information may be used to track the patient location and determine activity and behavior patterns. This additional information may include, for example, a priori knowledge of key locations in the home. This may allow such information to be determined as patient bedtime, average time in the bathroom, trips to the refrigerator, and door exits and entries, as well as other activity, route, and pattern-related information. Also, in one embodiment, the system and method may include, or operate in association with, a networked alert system provides notification to an interventionist, loved one, medical personnel, or other responsible party that a change has occurred in a risk status of the patient.

Whether located in the base station or a network-connected remote location, the processor of the monitoring system may generate the aforementioned metrics in order to detect or predict the condition of the patient, who either has already been diagnosed with COPD or who is a suspected candidate of COPD. The activity patterns, behavior patterns, and other metrics computed by the model(s) of the monitoring system may serve as an indicator of the presence or deteriorating condition of COPD.

The model(s) of the monitoring system may use the metrics as a basis for predicting hospital readmission. For example, the model(s) may be trained to indicate that hospital readmission is likely or imminent when one or more predetermined metrics lie in respective ranges or have certain values. The ranges and values may be determined, for example, based on training data that indicate the activity patterns, behavior patterns, vital sign data, and/or other indicia that are indicative of a likely or immediate COPD-related event.

In one embodiment, the patient may wear a tag (e.g., RFID tag) which communicates with anchors or beacons at various locations in the home. The signals generated by the tag and anchors/beacons may be used as a basis for determining the location of the patient in real time. As the subject inevitably and routinely traverses common pathways in the home (e.g. kitchen to couch, couch to bathroom, bedroom to bathroom, up and down stairways), the ratio of the pathway distance to time-to-traverse these pathways is recorded. Significant changes in mobility indicators may be announced by the alert system. Furthermore, the daily indoor mobility (e.g., stair usage, climbing speed, etc.) of the patient may be tracked and combined by the model(s) with physiological data (e.g., heart rate, respiratory rate, etc., captured by patient-worn sensor(s)) to make COPD-related predictions. In one embodiment, the patient-worn sensors may be integrated in the IPS tag worn by the patient or may be separate from such a tag. The aforementioned information may be stored and processed to determine COPD deterioration and may (e.g., with the use of a smart system) generate or otherwise determine reference values, ranges, thresholds, or a combination of outliers that indicate risk. These reference values, ranges, etc., may be determined, for example, based on an average measurement of historical patient data, a median of the historical data, or another mode or other statistical measurement. In one case, for example, when the risk is severe, the alert system generate and transmit a notification through a network to a person who may intervene for the patient to improve his/her health outcome.

In one implementation, the mobility indicator may be a metric that includes information indicative of 4 meter gait speed (4MGSP). Especially among the elderly, 4MGS may predict survival in large cohorts. For a COPD application, 4MGS may correlate with aspects of disease phenotype and may also indicate improvement or deterioration from pulmonary rehabilitation and prediction of the risk of readmission in patients with COPD after acute exacerbation. In one embodiment, the physiological measurements may be combined with mobility measurements (e.g., which may or may not include gait speed) to provide additional metrics indicative of the effect that walking has on the physiology of the patient. This combined metric may resolve or eliminate potential confounding factors, e.g., a reduction in gait speed due to undetectable events (patient multitasking while walking, urge to reach the destination, etc.). For instance, information indicative of similar walks may be clustered by similarity of route and gait speed, and this information may be combined with one or more types of physiological data (e.g., detected differences in heart rate and/or respiratory rate changes due to walking) may correlate to changes in the physical ability of the patient, which, in turn, may be identified as a risk factor for COPD detection or deterioration.

In one embodiment, to avoid false negatives a digital filter of the daily collected data may be used or the alert system may be implemented in a manner that requires successive data outliers to be detected before a notification is triggered and transmitted.

### IPS System Features

In one embodiment, the indoor positioning system (IPS) may include a patient-worn tag that communicates with beacons placed in the home. The tag and beacon information may be processed by a positioning algorithm to determine the indoor location of the patient at all times. The tag and beacon may operate, for example, at one or more frequencies that exceed 40 Hz. In one embodiment, the tag and beacons may transmit/receive ultra-wide band (UWB) radio signals that allow the patient location to be determined with sub-centimeter accuracy, even with non-line-of-sight (NLOS) measurement (e.g., through walls).

The number of beacons placed in the home may be determined, for example, based on the number of measurement factors (e.g., metallic objects, multipath interference, accuracy specifications) in the coverage area and system costs. The United States, European Union and several Asian pacific countries have allowed for unlicensed commercial use of the UWB frequencies between 3.1 and 10.6 GHz, provided the transmission are compliant with the National Telecommunication and Information Administration.

In one embodiment, patient location may be determined based on Time of Arrival (TOA) information, Time Difference of Arrival (TDOA) information, Angle of Arrival (AOA) information, or Relative Signal Strength (RSS) information. The transmitted and received signals may be made more robust in the presence of interference using several techniques. Examples of these techniques include signal modulation, Pulse Width Modulation (PWM), Pulse Amplitude Modulation (PAM), or on-off keying (OOK). Furthermore, in order to minimize the effect of multipath ambiguity (e.g., a phenomenon commonly occurring when signals bounce off of objects and are received multiple times), Time hopping spread spectrum (TH-SS) radio impulses or binary phase shift keying (BPSK) may be used.

In one embodiment, filtering may be employed by the receiver to determine true location using a series of measurements for one location weighted least squares multi-dimensional scaling or extended Kalman Filters. Although in one embodiment of an IPS incorporates UWB signaling, another embodiment may use a different type of signaling including, but not limited to, infrared (IR), WiFi, LiFi, Bluetooth (BT), Bluetooth Low Energy (BLE), imaging (camera), RFID, NFC, cellular devices, magnetometers, accelerometers, gyroscopic meters, inertial meters, floor pressure sensors, satellites, and pseudolites. Also, in one case, a hybrid-type IPS system may be used which combines various forms of signaling previously mentioned.

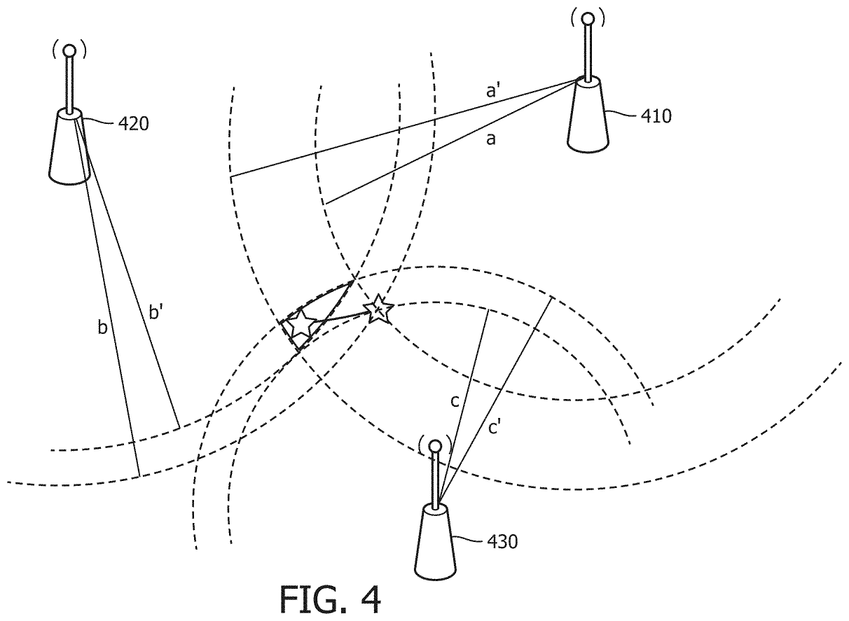

FIG. 4 illustrates an example of how deviation of computed location-related metrics from one or more reference locations may be detected or predicted by the processor of the monitoring system as a COPD-related event. In this example, three beacons placed at predetermined locations throughout the home of the patient. For example, beacon 410 may be in the kitchen, beacon 420 may be in the bathroom, and beacon 430 may be in the living room.

Each beacon has associated with it at least one reference range. For example, beacon 410 may have a reference range indicated by distance a and line 411, beacon 420 may have a reference range indicated by distance b and line 421, and beacon 430 may gave a reference range indicated distance c and indicated by line 431. These reference ranges may be considered to define normal movement or position ranges of the patient, as determined, for example, based on historical or training data for the model implemented by the processor of the monitoring system. Additionally, each beacon may have associated with it at least one outlier range. For example, beacon 410 may have a first outlier range denoted by distance a′ and line 412, beacon 420 may have a second outlier range denoted by distance b′ and line 422, and beacon 430 may have a third outlier range denoted by distance c′ and line 432. The location of the patient may be determined based on a patient-worn location tag transmitting a signal (e.g., UWB pulses) relative to the beacons.

The difference between the reference and outlier ranges may define a deviation amount (Δ) that provides a basis for determining or predicting a COPD-event with a first probability. For example, in one embodiment when the location of the patient is between lines 421 and 422 corresponding to the reference and outlier ranges, the model may predict that a COPD-event exists with a first probability. When the location of the patient has exceeded the outlier range, then the model may predict that a COPD-event exists with a second probability greater than the first probability. The probabilities may correspond to risk states or risk scores. In order for a risk state or score to be determined, one implementation may require the patient to be at a location which exceeds the reference or outlier ranges for at least a predetermined period of time, for example, in order to filter out possible false positives.

In one embodiment, a patient may not be identified to be in a probabilistic risk state (or may have a risk score of 0) if the patient is at a location which has exceeded the reference or outlier range relative to only one beacon. In this case, to be considered in a risk state or to have a non-zero risk score, the location of the patient must simultaneously exceed the reference range or the outlier range relative to at least two of the beacons (or in some cases all three beacons). Such a situation is exemplified by the changing positions of a star 450 in FIG. 4. For example, in FIG. 4, star 450 marks the location of the patient at an initial position 470, which coincides with the intersection of the lines delimiting the reference ranges for beacons 410, 420, and 430. Star 450 is at a second location 480 to mark movement of the patient from the initial position. Second 480 location exceeds reference range 411 of first beacon 410 but not its outlier range 412, exceeds reference range 421 of second beacon 420 but not its outlier range 422, and exceeds outlier range 432 of third beacon 430.

When the position of the patient is at second location 480 (marked by the changed location of star 450) for at least a predetermined period of time, then the combination of relative ranges from respective ones of the beacons is identified by the processor of the monitoring system as a risk factor, warranting prediction that the patient is experiencing a COPD-related event. In one or more embodiments, a COPD event may be considered to correspond to detection or prediction of COPD in the patient, detection or prediction of a deteriorating COPD condition of the patient, and/or another event related to COPD.

The UWB pulses transmitted from the patient-worn tag may be at a high carrier frequency. At this frequency, low-speed physical activity of the patient may be detected with a certain accuracy and also may allow for oversampling and adequate speed measurement of the patient in the home, e.g., greater than 40 Hz update rate. Furthermore, the low-power requirements of a UWB transmitter and receiver may allow the system to operate for an extended period of time (e.g., more than 1 year) when powered by a low-density battery.

In one embodiment, a memory storage device may be coupled to or otherwise in communication with a central transmitter, a selected one of the anchors/beacons, or the base station (which may correspond to the central transmitter or selected anchor/beacon) in order to store the sensor data generated by the daily route and other information and the metrics generated for the patient, as well as floor plan or map information of the house including 3D coordinates of stairs in the patient home.

A microprocessor may be included inside a master anchor (or base station) to perform the route calculation and to store the daily activity and gait speed metrics. Mean metric computing logic may be included inside the microprocessor to determine the average coverage area percentage (e.g., heat map), average number of stair traversals, average daily climbing speed (associated with stairs), average daily descending speed (associated with stairs), number of common pathways and the daily average gait speed associated with the common, and/or other metrics over predetermined periods of time, e.g., hourly, daily, in real-time, etc.

In one embodiment, microprocessor or other processing logic (e.g., processor 210) generating and processing the sensor data and/or metrics may be located outside of the home. In this case, the sensor data may be transmitted to the processor of the monitoring station, for example, periodically or in real-time in order for associated metrics to be generated and/or interpreted as a basis for detecting a COPD-related event.

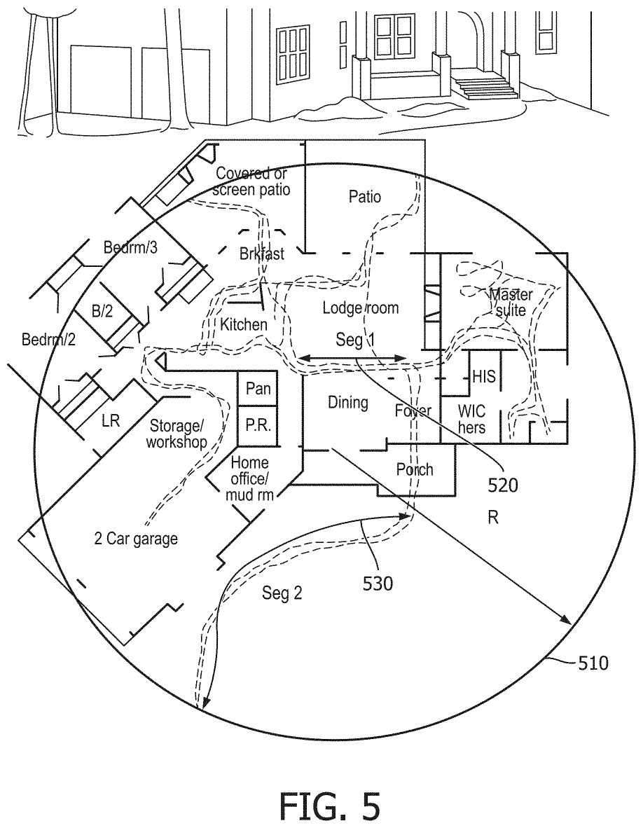

FIG. 5 illustrates an example of how the system and method may generate routing information superimposed or relative to a house map. In this case, the processor of the monitoring system (or base station, or both) generates routing information superimposed on a two-dimensional floor plan of the house of the patient.

Referring to FIG. 5, the routing map includes a perimeter 510 indicating the range R of the IPS and/or other sensors used to determine the location and track routes and paths taken by the patient over a predetermined period of time, e.g., one day. The routing map includes two types of routes. The first type of routes 520 are daily location routes taken by the patient as he traverses through the kitchen, lodge room, bathroom, and other rooms of his house. These routes may be considered normal routes and therefore may be used, for example, to establish a baseline for training data to be used as a model implemented by the monitoring system processor for detecting or predicting a COPD-related event. The second type of routes 530 are common pathways traversed by the patient throughout and around his house. In operation, the processor may detect significant deviations from the daily location routes and/or common pathways in order to detect a COPD-related event. The deviations may be based on, for example, one or more distance threshold values or a location (e.g., room or outside area) where the patient seldom, if ever, goes.

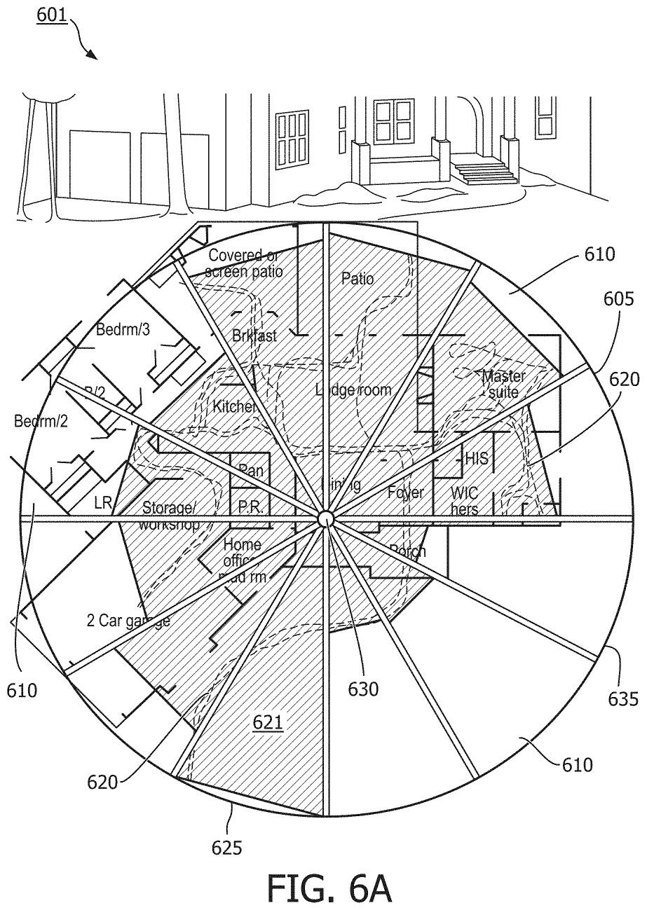

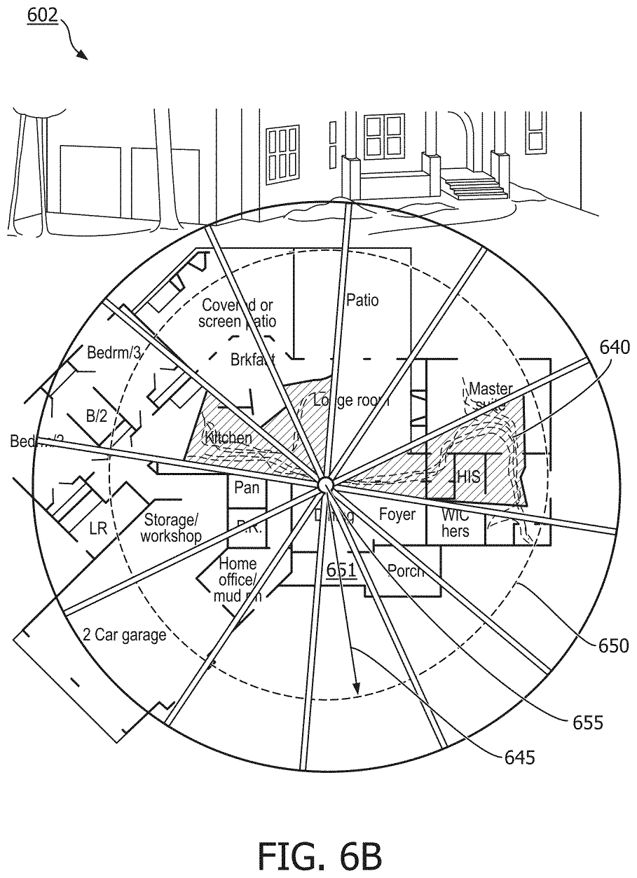

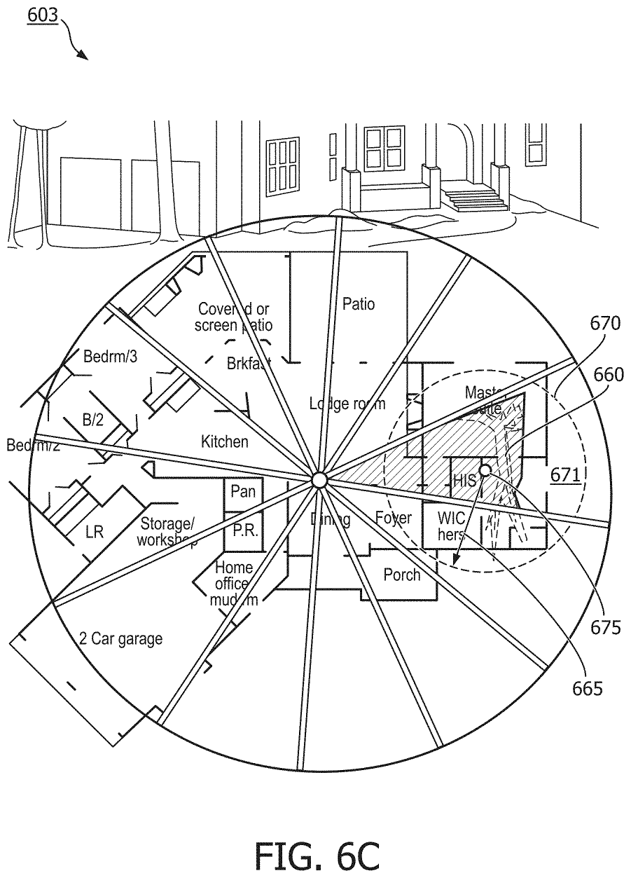

FIGS. 6A to 6C illustrate examples of how the system and method may generate heat maps that indicate the location of patient, each throughout a predetermined or specified time period, e.g., a day. The processor of the monitoring system (or base station, or both) may generate the heat maps based on the routing information (and/or other IPS or location/tracking information) derived from the sensors arranged in the house of the patient.

FIG. 6A illustrates an example of a heat map 601 that reflects a normal day of activity and behavior on the part of the patient. The heat map includes zone designations superimposed on the floor plan of the house of the patient. The zone designations may be in the form of sectors 610 allocated within the perimeter 605 corresponding to the range of the sensors. The sectors may be allocated, for example, on the basis of rooms of the house or may corresponding to a predetermined template or pattern. In the case of FIG. 6A, the sectors are arranged within the confines of a circular template.

The heat map conveys various types of information, not the least of which includes coverage areas within the house that the patient may have, or may have most often, frequented within each sector. In this case, the heat map is generated based on the daily location routes 620 illustrated in FIG. 5, and may include a perimeter 605 defined by the most distant point of the route in the routing map that is most distant from a reference point (e.g., center or another point) 630 of the map template. In order to generate the map content, the processor of the monitoring system (or base station) determines intersections between the routes and the sectors. When one or more of the routes overlap (or otherwise reside in) a sector, that sector is shaded. Sectors which does not include any routes are not shaded. Thus, the heat map conveys information indicating, on a coverage area basis, the locations of the patient throughout the day (or other time period). The coverage area 621 in the heat map of FIG. 6A may correspond to one or more, or all of, the shaded areas.

In one embodiment, the shading in each sector of the heat map may be partial, corresponding only to the areas of that sector which includes the routing information. Thus, for example, sector 625 is almost completely shaded because it includes at least one route that spans throughout the entire sector. In contrast, sector 635 only has partial shading because the patient only traversed a route that only intersected a small area of this sector. Because the heat map in FIG. 6A reflects normal activity patterns of the patient, the shading in the sectors of this heat map may be used as a reference or baseline for determining activity patterns that may be indicative of a risk or COPD-related event.

FIG. 6B illustrates an example of a heat map 602 generated when the activity of the patient has deviated from the normal (or reference) activity pattern (e.g., exemplified by the heat map in FIG. 6A) by a first level, e.g., a moderate amount. In heat map 602, daily location routes 640 are confined to a much smaller area of the house. As a result, radius 645 and perimeter 650 of the heat map template are much smaller, extending out to the most distant point of the most distant route from a reference point 655 (e.g., center or another point) of the map. In one embodiment, the location of the reference point may be shifted based on the locations of the routes and in order to generate the map with the smaller radius and perimeter. Because of differences in the routes and sizes of the radius and perimeter, the location of reference point 655 in the heat map of FIG. 6B is different from the location of reference point 630 in the heat map of FIG. 6A. In another embodiment, these references points may be coincident, for example, depending on the locations of the routes. Also, in the heat map of FIG. 6B, there are fewer routes and shaded areas, which reflect diminished physical movement on the part of the patient throughout the period of observation.

Once this heat map has been generated, it may be compared by processor 210 of the monitoring system to the heat map in FIG. 6A and/or to other reference information. For example, the processor may compare the degree of shading (e.g., shading on a percentage basis relative to the floor plan of the house), the location of the routes and shading (or omissions of routes or shading in areas that the patient is commonly expected to occupy throughout the day), and/or the perimeter and/or radius size (any one or combination of which may referred to as coverage area 651) to the shading, radius size, perimeter size, routes, and/or other similar information (e.g., coverage area) in the heat map of FIG. 6A. Based on this comparison, the processor may determine that the deviation in patient activity lies in a predetermined range or exceeds a predetermined threshold (e.g., in a first probability range) indicative of moderate or first level of risk of a COPD-related event.

FIG. 6C illustrates an example of a heat map 603 generated when the activity of the patient has deviated from the normal activity pattern (e.g., exemplified by the heat map in FIG. 6A) by a second level, e.g., a very significant amount. In heat map 603, daily location routes 660 are confined to a very small area space of the house. As a result, radius 665 and perimeter 670 in this heat map is much smaller, extending out to the most distant point of the most distant route from a reference point 675 (e.g., center or another reference point) of the map. In this embodiment, the location of the reference point is shifted based on the locations of the routes and in order to generate the map with the smaller radius and perimeter. Because of the size differences caused by the diminished activity, the location of the reference point in the heat map of FIG. 6C may not coincide with the locations of either one of the reference points in the heat maps of FIGS. 6A and 6B. Also, in the heat map of FIG. 6C, there are far fewer routes and shaded areas relative to the floor plan (e.g., and/or the affected sectors), which reflect minimal physical movement on the part of the patient.

Once the heat map has been generated, the processor of the monitoring system may compare the degree of shading (e.g., shading on a percentage basis relative to the floor plan of the house), the location of the routes and shading (or omissions of routes or shading in areas that the patient is commonly expected to occupy throughout the day), and/or this perimeter and/or radius size (any one or combination of which may be referred to as a coverage area 671) to the perimeter and/or radius size to the shading, coverage area, radius size, perimeter size, routes, and/or other similar information (e.g., coverage area) in the heat map of FIG. 6A, or on a relative basis to this information in the heat map of FIG. 6B (which, for example, may be stored and retrieved for comparison purposes). Based on this comparison, the processor may determine that the deviation in patient activity lies in a predetermined range or exceeds a predetermined threshold (e.g., in a second probability range greater than the first probability range) indicative of very serious or life-threatening risk of a COPD-related event.

In one embodiment, in all of the heat maps the reference point may correspond to a median coverage point relative to the area traversed by the specific routes. In another embodiment, the reference point may correspond to a central point of the house or floor plan. In another embodiment, depending on placement of the routes, one or more of these reference points may be coincident.

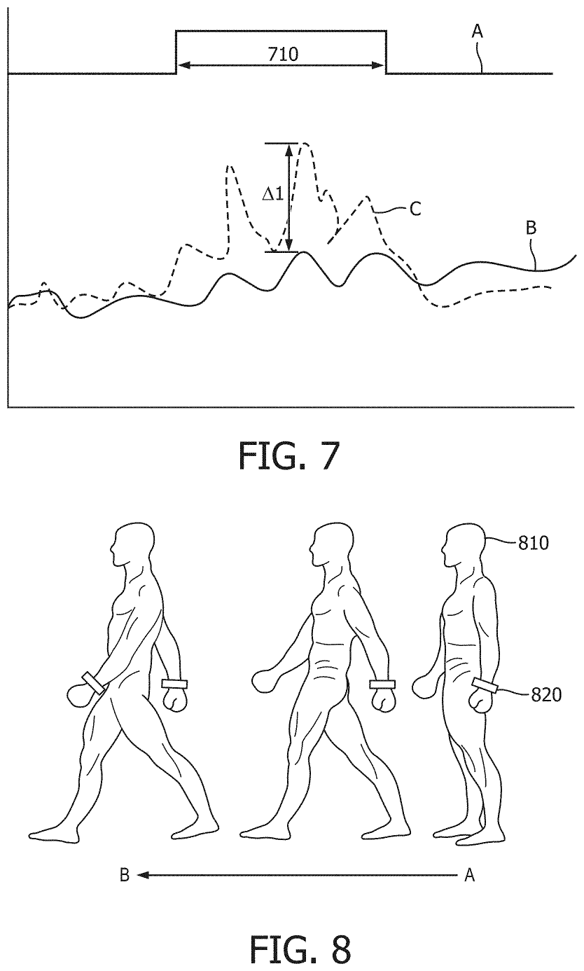

FIG. 7 illustrates another example of how the processor of the monitoring system, through execution of one or more algorithms or models, for example, as previously described, may compute a deviation that corresponds to a COPD-related event. In this example, the processor receives first information that indicates or tracks patient location and second information that indicates or tracks the posture, position, orientation, or movement of the body or a body part of the patient during the observation period. The first and/or second information may correspond to any one or more of the metrics described herein.

In this example the first information corresponds to detection of movement as the patient climbs a staircase and the second information corresponds to arm movement as the patient walks, as detected, for example, by a patient-worn sensor such as a wrist monitor. The determination of a COPD-related event is performed by based on a correlation of this information in comparison to reference information, e.g., generated based on training data for the patient or generic patterns of a healthy patient.

Referring to FIG. 7, the first information corresponds to curve A showing in section 710 a period of time when the patient is detected as climbing the stairs, e.g., based on three-dimensional information obtained by beacon or tracking monitors positioned in the house. The second information corresponds to curve B which has been superimposed on reference information in the form of curve C. The reference information of curve C shows a substantially uniform pattern of arm movement has a healthy person (e.g., a person without COPD) climbs stairs. In contrast, the curve C demonstrates an erratic pattern of arm movement relative to curve B. The processor may compute the deviation (Δ1) between the curves during the period in which the patient is detected as climbing the stairs to determine a COPD-related event.

In one embodiment, the deviation may be computed based on a mean amplitude of curve B relative to a mean amplitude of curve C. If this deviation exceeds a predetermined threshold value, then the processor of the monitoring system may generate a signal indicating a COPD-event is likely. In other embodiment, the deviation may be computed based on a difference between the highest peak value of curve B and the highest peak value of curve C. In other embodiments, the deviation may be computed using another method. Also, in other embodiments, the first information and the second information may be any one or more, or combinations, of the types of metrics described herein, including but not limited to vital sign information.

In one implementation, three types of information may be used as a basis for detecting a COPD-related event. The three types of information may include, for example, the first and second information described above, coupled with respiratory rate information generated from another patient-worn sensor. The respiratory rate information may be processed by the monitoring system to determine a metric such as, for example, a waveform indicative of changes in respiratory rate of the patient relative to patient movement, tracking, location, or during other activity taking place during the observation period. When the respiratory rate metric exhibits a certain predetermined pattern (e.g., erratic pattern, increased amplitude pattern, etc.), for example, during the time when the arm movement pattern is erratic in the stair-climbing period, then the processor of the monitoring system may determine that the patient is experiencing a COPD-related event.

### Additional Embodiments

In one embodiment, the metric that may be used to detect or predict a COPD-related event may be the gait of the patient. The gait of the patient may be determined, for example, based on information generated by a position tag worn on the wrist of the patient. This position tag may include, for example, an accelerometer and/or other sensor for determining the arm swing movement of the patient, which, in turn, may provide an indication of the gait of the patient.

More specifically, analytics of a wrist-worn position tag may provide a number of predictive elements to determine pathological gait and increased morbidity in patients having a chronic disease. While the specific case of COPD is discussed here, the disease may be a condition different from COPD in another embodiment. Different pathological gait patterns may arise from a variety of deformities, including but not limited to musculoskeletal weakness, neurological disorders, or trouble during proprioception. The sensor(s) in the tag may detect these patterns and transmit corresponding information to the base station or monitoring system using RF or other types of signals. The signals may be transmitted from the tag, for example, through a wireless Bluetooth, WiFi, or other type of communication link or network connection.

FIG. 8 illustrates an example of a patient 810 walking with a position tag 820 on the wrist of his left arm. In this example, the patient walks from position A to position B. As depicted, it appears that the patient takes only two steps to get to position B. However, it is understood that in some embodiments a number of additional steps may be taken between A and B. For example, in one case position A may be in one room and position B may be in another room of the house of the patient, where many steps are required to go from A to B. In another case, position A may be on one floor of the house and position B on another floor, thus requiring the patient to descend or ascend a set of stairs to get from A to B.

In traversing from position A to position B, the sensor(s) in the wrist tag may determine a time series of wrist position coordinates that may be indicative of arm swing metrics (which, for example, may be indicative of happy walk, frail walk, etc.), shoulder movements, and/or sway due to circumduction of the foot or imbalance. The following position data recorded in three directions (x, y, z) may provide an indication of these gait-related parameters/metrics.

| Time(sec) | X (m) | Y (m) | Z (m) |
|---|---|---|---|
| 0 | 0 | 2.0 | 0 |
| 0.5 | 0.3 | 2.1 | 0.1 |
| 1.3 | 1.7 | 2.2 | 0.1 |

In one embodiment, an arm swing pattern may be determined based on the position data derived from the wrist-worn tag measured over time. Brisk walking may be indicative of a healthy patient and may correspond to movement of the arms in a pendulum pattern. Conversely, walking in a stooped over position with limited or erratic arm movement may be indicative of a frail patient who may be experiencing a COPD-related event. In more extreme examples (e.g., Hemiplegic or Diplegic gait), walking is characterized by flexion of arms in a locked position with no wrist movement. Frail walking that results in a flexion of the upper body may be characterized by a Parkinsonian gait, which limits arm movements while the patient takes small steps known as “Marche a petits pas” walk of little steps.

The arm swing of a patient may be quantified, for example, by generating a time series of coordinates in a minimum of two dimensions based on the tag sensor data. The time series of coordinates may then be compared by the processor of the monitoring system with the floor plan of the patient house or abstractly in any two-dimensional space. The coordinates may then be stored in time-series form as the patient traverses from point A to B.

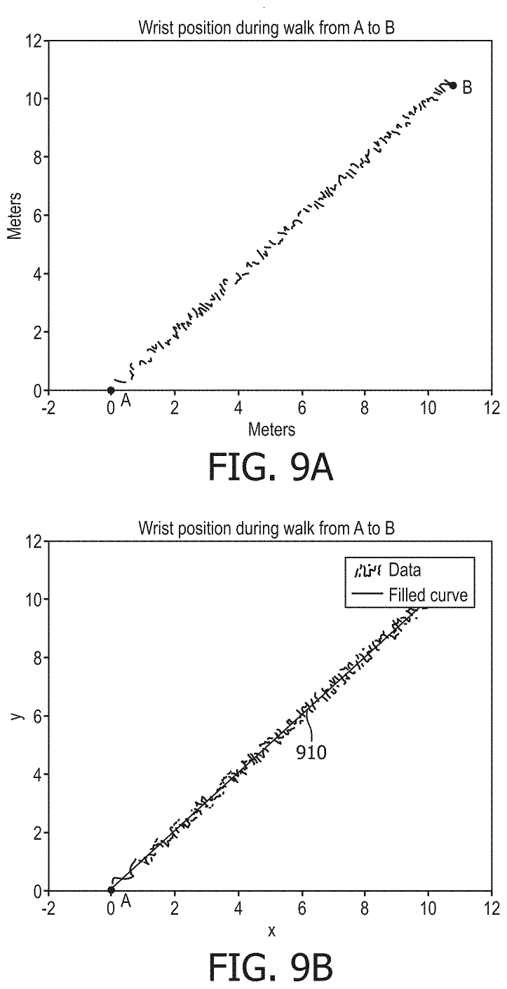

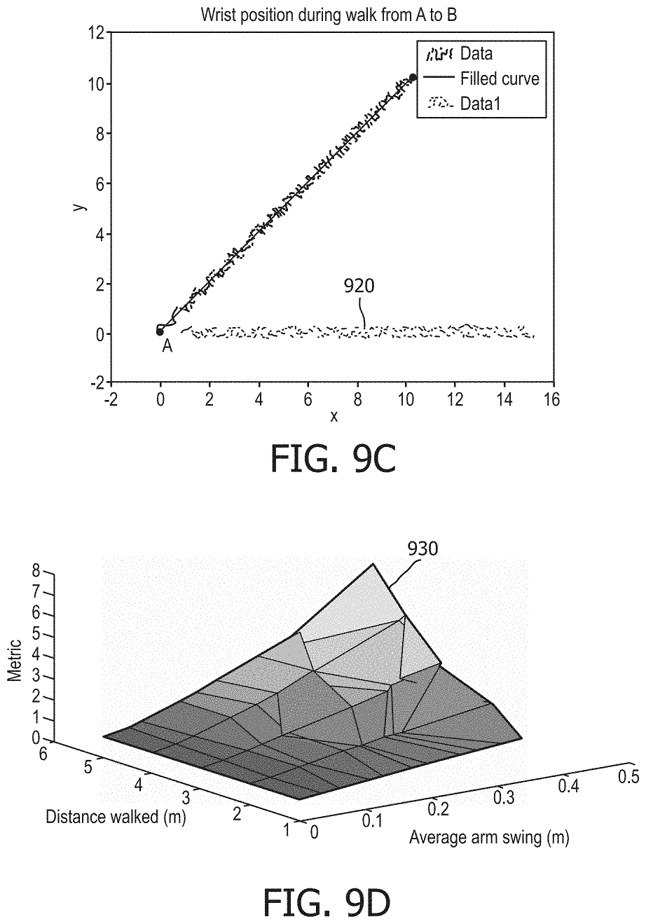

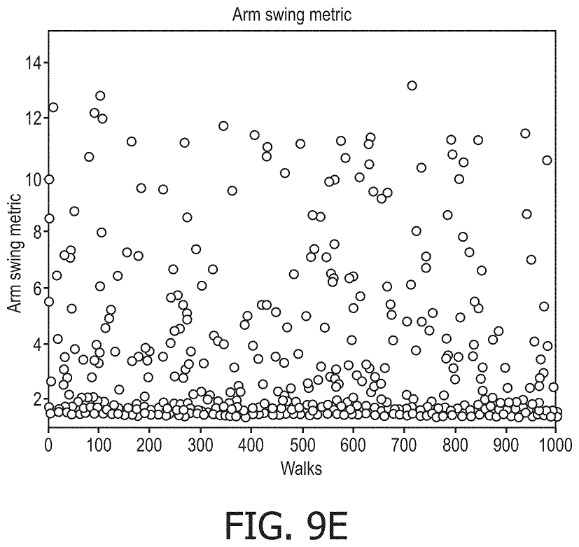

FIG. 9A illustrates an example of a graph of position data generated by the tag as the patient walking from position A to position B. The position data measured by sensor(s) in the tag correspond to the black dots and represent measurements taken in two dimensions (e.g., x-y, x-z, or y-z). Here, the position is sampled every 100 msec. and the point corresponding to position A is arbitrarily assigned to be at the origin by subtracting the coordinate of point A from each measurement.

The tag sensor data in the graph may be processed to derive metrics of arm swing during the walk. In one embodiment, this may be accomplished by fitting the data to a line. The data is contained in the time-series positional vector, P from P_{A} to P_{A}, where two new variables, X and Y, are assigned as follows:

$$X = \vert 1 \begin{matrix}\text{?} \\ \text{?}\end{matrix} \vert , Y = \vert \begin{matrix}\text{?} \\ \text{?}\end{matrix} \vert . \\ \text{?} \text{indicates text missing or illegible when filed}$$

Using linear least squares, a linear model \hat{β} may be generated where:

\hat{β}=(X^{T}X)⁻¹X^{T}Y.

In this equation, the second element of \hat{β} represents the slope of the best fit line. This slope may be represented as the angle θ of the direction away from the x axis, where this angle may be given by:

θ=tan⁻¹(\hat{β}₁)

FIG. 9B illustrates a graph that includes a line 910 that corresponds to the best fit line represented by the data of wrist position during the walk. In order to simplify the arm swing metric, a translation may be performed which includes rotating all of the data onto the x axis using the angle of the slope calculated by the above equation and then performing a rotational transformation given by the following matrix:

P¹=|sin [symbol] cos [symbol] 0|·P

FIG. 9C illustrates a graph showing an example of these operations. In this graph, line 920 (corresponding to data1) represents translation of the positional data onto the x axis. Once this translation has been performed, the arm swing metric may be computed by using the ratio between the distance walked and the sum of the differences in the x direction between all green points, as indicated by the following equation:

$${δ}_{{arm}_{-} s w i n g} = \frac{\sum ( {P}_{{x}_{i}}^{′} - {P}_{{x}_{i - 1}}^{′} )}{distance walked} ,$$

The distance walked may be given by the following equation:

distance walked=\sqrt{(P_{B}_{_{y}}−P_{A}_{_{y}})²+(P_{B}_{ₓ}−P_{A}_{ₓ})²}

Using this metric, it is evident that patients with healthy arm swings have large arm swing metrics, while frail walking with flexed arms have a δ_{arm_swing} that approaches 1. When expressed as a ratio, this metric is less sensitive to the distance walked, but the ratio increases slightly with longer walks. This gives a boost in the health indicator when the patient is walking longer distances, but in general, the ratio reflects movement of the arms.

FIG. 9D illustrates a surface plot showing data taken over a variety of walks, with multiple measurements of the arm swing metric. For example, the surface plot shows that the metric grows significantly as the arm swing amplitude is increased, but the increase as a function of distance walked is more subtle. The upper triangular section 930 in the surface plot may be considered as healthy, happy walking with good arm movement. The metric δ_{arm_swing} may be used regularly (e.g., each day) as a health monitor because it indicates the degree of arm swing corresponding to healthy brisk walking and the distance covered.

FIG. 9E illustrates an arm swing metric generated based on data for the last 1000 walks. The metric could be analyzed to detect the following indicators of health decline or neurological impairment that results in the flexion of the upper body or Parkinsonian shuffling.
1. Downward trend over time
2. Lower percentage of walks per day above a “healthy” threshold, e.g., 4.0
3. Anomaly detection method.
   a. (Density Method) Daily arm swing metric is located in a low-density area.
   b. (Distance Method or K Means) Daily arm swing metric is different from the mean by using K means clustering techniques.
   c. (Parameterization) The Daily arm swing metric is modeled over the past several days and according to a parameter related to the slope of the line, the slope is more negative.
   d. (Isolation) Using isolation forests, the Daily arm swing metric is an outlier because it is isolated by a lower number of edges using isolation decision trees.

### Sway Metrics Embodiments

In one embodiment, the sway of the patient may be used as a metric to detect or predict a COPD-related event. Sway is a symptom characterized by a clumsy staggering gait, which may be indicative of a problem in the brain (e.g., cerebellar or ataxia) that causes coordination or balance issues. In COPD, sway may also result from unsteadiness that occurs by exertional dyspnea, that tends toward the patient leaning upon walls while moving, grabbing for supporting structures or stumbling from chest tightness. A patient walk that has signs of sway may also be detected by a wrist-worn positional sensor.

The sway metric may be generated, for example, by acquiring the time-series positional vector P as the patient walks from position A and position B, as previously described. As before, all of the data may be referenced to the origin at position A.

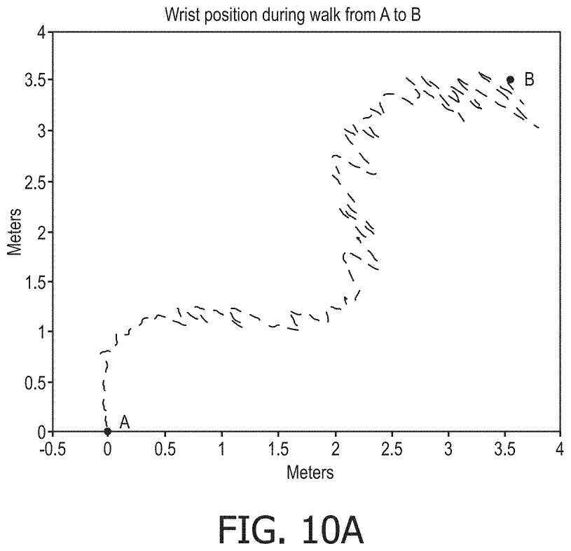

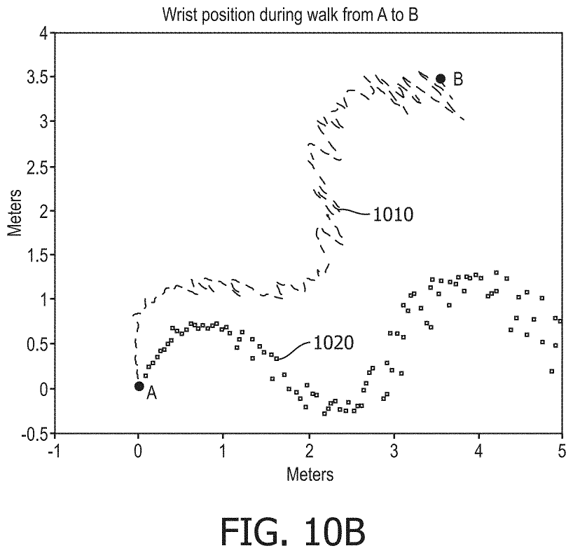

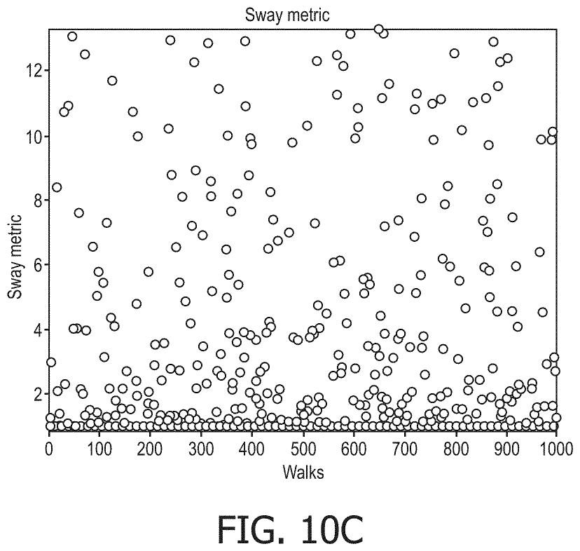

FIG. 10A is a graph illustrating two-dimensional (e.g., x-y) data generated by a patient that exhibits a sway pattern during a walk from position A to position B. For sway detection, the process of fitting a line may be inaccurate under some circumstances. Thus, in one embodiment, the angle of walk may be determined simply by the two endpoints, for example, based on the following equation:

$$θ = t a {n}^{- 1} ( \frac{{P}_{{B}_{Y}}}{{P}_{{B}_{X}}} )$$

The data may be translated onto the x axis in a manner as previously described, for example, in accordance with the following translation matrix:

$${P}^{′} = [ \begin{matrix}cos ( θ ) & - s in ( θ ) & 0 \\ sin ( θ ) & cos ( θ ) & 0 \\ 0 & 0 & 1\end{matrix} ] \cdot P$$

FIG. 10B illustrates a graph showing the untranslated data 1010 and the data 1020 translated onto the x axis using the above matrix. The sway metric may be computed according to the following equation based on the ratio between the distance walked and the sum of the differences in the y direction between all of the points 1020.

$${δ}_{{arm}_{swing}} = \frac{\sum ( {P}_{{y}_{i}}^{′} - {P}_{{y}_{i - 1}}^{′} )}{distance walked}$$

where the distance walked may be given by:

dist|ance walked=\sqrt{(P_{B}_{_{y}}−P_{A}_{_{y}})²+(P_{B}_{ₓ}−P_{A}_{ₓ})²}

FIG. 10C illustrates a plot showing the sway metric computed over the last 1000 walks taken by the patient. This metric may be analyzed to detect the following indicators of health decline or neurological impairment that results in staggering or swaying.
1. Upward trend over time
2. Lower percentage of walks per day below a “healthy” threshold, e.g., 4.0
3. Anomaly detection method
   a. (Density Method) Daily sway metric may be located in a low-density area.
   b. (Distance Method or K Means) Daily sway metric may be different than the mean by using K means clustering techniques.
   c. (Parameterization) The Daily sway metric may be modeled over the past several days and according to a parameter related to the slope of the line, the slope is more positive.
   d. (Isolation) Using isolation forests, the Daily sway metric may be an outlier because it is isolated by a lower number of edges using isolation decision trees.

In one embodiment, a stair-climbing pattern may be used as a metric to detect or predict a COPD-related event of the patient. Data for generating the metric may be derived from a wrist-worn position tag, for example, as previously described. A stair-climbing pattern may be used to detect COPD because COPD patients may have difficulty climbing stairs.

More specifically, when a patient has pulmonary problems, the patient may try to avoid climbing stairs and many other forms of simple exercises that have proven to be beneficial to long term health and well-being. A patient with severe COPD may become breathless with very uncomfortable chest tightening when ascending stairs. This physiological response may tend to discourage the patient from using the stairs. With a wrist-worn positioning tag, a determination may be made as to the performance of the patient and usage of stairs within the house on a regular (e.g., daily) basis. The data may be taken over time to generate a time series of data that may be used as a basis for estimating deterioration in the COPD condition of the patient.

For stair-climbing metric, the processor of the monitoring system may interpret data from a wrist-worn position sensor with location information (e.g., a blueprint or floor plan) of the house. The data includes known 3D-coordinates corresponding to areas of the house that include steps or stairs. In some cases, the floor plan or house location/layout information may indicate where a banister is located on the stairs (e.g., left or right or both sides) relative to the stairs. The positional data may be used as a basis for determining the presence and use of a banister. Use of a banister or a handrail may also be determined from the sensor data when the position tag is placed on the wrist that grasps the handrail while ascending or descending the stairs.

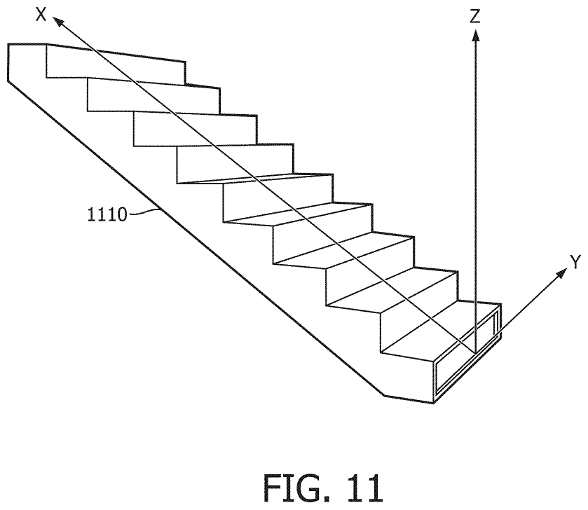

FIG. 11 illustrates an example of a set of stairs 1110 in the house of a patient wearing a wrist monitor. In this example, position A is located at one end of the stairs and position B may be located at an opposing end of the stairs. In traversing from position A to position B up or down the stairs, the time series of wrist position coordinates may determine the following:
1. Time of going up or down a set of stairs
2. The use of a hand railing while going up or down stairs
3. Number and Duration of pauses on the steps indicating a need for respiratory recovery
4. The number of steps taken per day

In interpreting this data, a coordinate system (as shown) may be defined relative to the stairs. In one embodiment, the coordinate system may correspond to a dimensional system in which the primary component (x) is parallel to the direction of movement of the patient upon the set of stairs, and the other two dimensions (y and z) are arranged orthogonally relative to the primary direction, with the z coordinate representing height.

In one embodiment, the information coordinates of the data generated from the wrist-worn sensor may be rotated (or otherwise translated) to any system of three orthogonal axes to improve processing efficiency or to obtain more relevant information. The horizontal direction (x) and many of the other metrics may then be derived based on a time series of one- or two-dimensional data sets.

As COPD disease becomes more severe, the time required to ascend the stairs increases. In some cases, a patient with a mild COPD condition may have significantly lower times in clinical performance testing measuring their ability to ascend 12 steps, while a significant difference may not be noticed (e.g., from data derived from treadmill walking) between mild and severe patients. The total time to traverse the complete horizontal distance of the stairs may be determined, for example, from the sensor data.

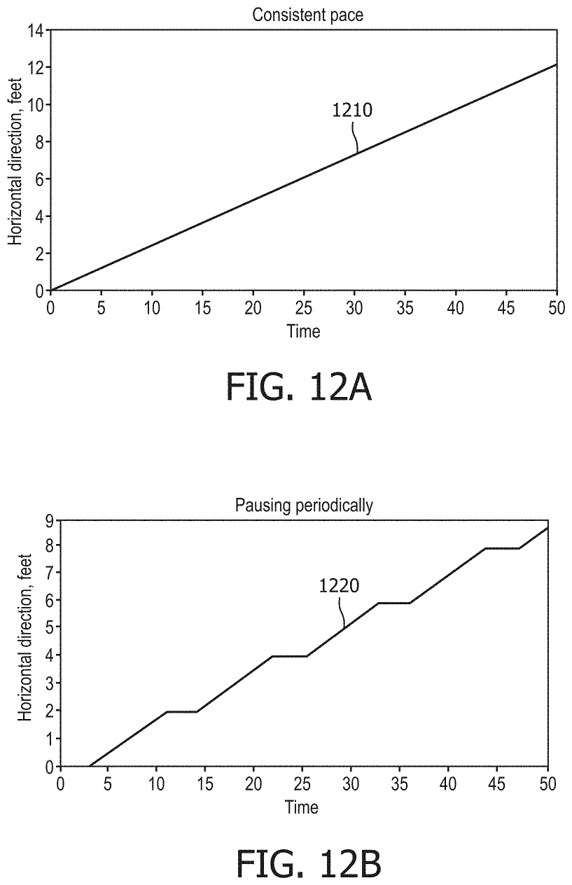

FIG. 12A illustrates a graph of a first example where the sensor data is plotted as a line 1210 indicating that the patient climbed the stairs in 50 seconds with a consistent pace. This graph indicates a patient with mild COPD or no COPD. In contrast, FIG. 12B illustrates a graph of a second example where sensor data is plotted as a line 1220 indicating a more erratic pattern for a patient who ascended a shorter set of stairs in 50 seconds, but had to pause four times during the ascent. In this latter case, the data is processed into a metric indicating a moderate to severe COPD condition.

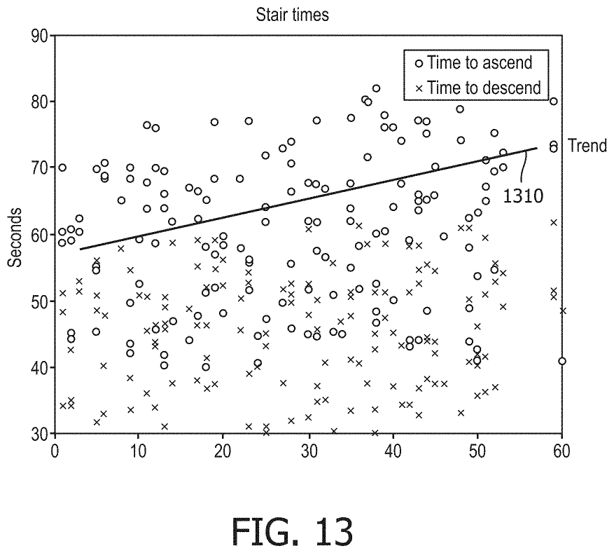

FIG. 13 illustrates an example of a graph plotting data points corresponding to two types of activity. The data points expressed as dots correspond to the patient ascending the stairs and the data points expressed as x's correspond to the patient descending the stairs. The data points are plotted against respective vertical and horizontal time axes. The processor of the monitoring system may process these data pints to generate a trend line 1310. The trend line shows an upward trend in multiple stair climbs over a 60 day period that may indicate that the COPD condition of the patient is worsening. Aside from trending these times, the stair-climbing metric may identify patients that have a significant change in the time it takes to climb or descend a staircase. This change may be detected using anomaly algorithms with statistics to determine the condition of the patient.

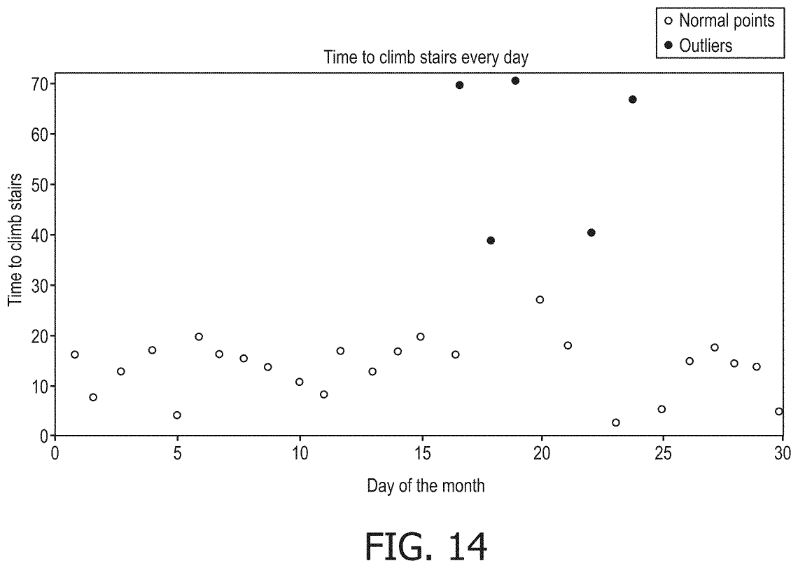

FIG. 14 illustrates a graph with data points derived from the wrist-worn sensor plotted against three axes. The first axis corresponds to the day of the month. The second axis corresponds to the time to climb the stairs. The third axis corresponds to the aggregate time to climb the stairs over a predetermined period of time, e.g., a daily basis. The data points (open circles) below the 30-second time on the first axis represent normal data points, while the data points above the 30-second time represent outliers, e.g., data points that are statistically outside a normal range developed for the patient. These outliers are of particular interest, in that an increase in the frequency and/or severity of the outliers may allow the processor of the monitoring system to determine a worsening COPD condition of the patient.

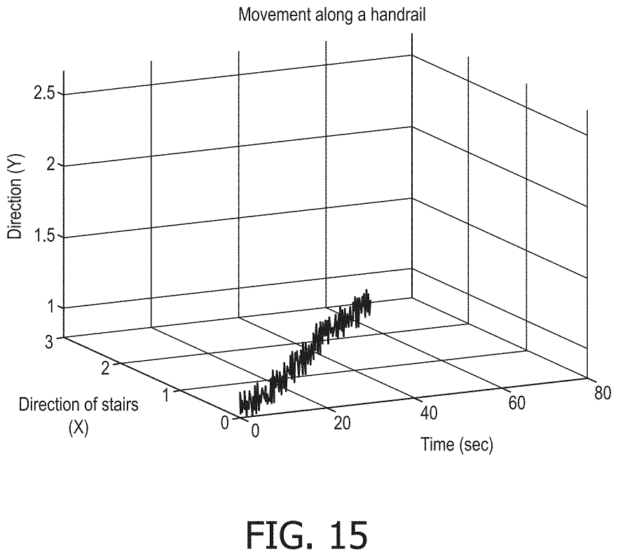

FIG. 15 illustrates an example of a three-dimensional plot of data generated by the processor of the monitoring system based on the wrist-worn sensor, in this case specifically relating to movement along the handrail of the stairs. The vertical axis of the plot corresponds to y-direction movement along the stairs, and the two horizontal axes respectively indicate x-direction movement along the stairs and the time to ascend or descend the stairs. When walking up and down the stairs, a patient may use the handrail to promote stability and aid in the ascension of the steps. This involves the use of upper body muscles. When the wrist tag is worn on the hand that is using the rail, the resulting motion tends to be uniform in the primary direction (x), with only small deviations from the direction of motion when the hand is temporarily lifted off the rail and repositioned. In the example shown in this plot, there is very little deviation in the y-coordinate of movement (e.g., 5 cm max deviation) as computed by the processor. From this, the processor may determine that the hand of the patient is moving along the banister only in the direction of the stairs.

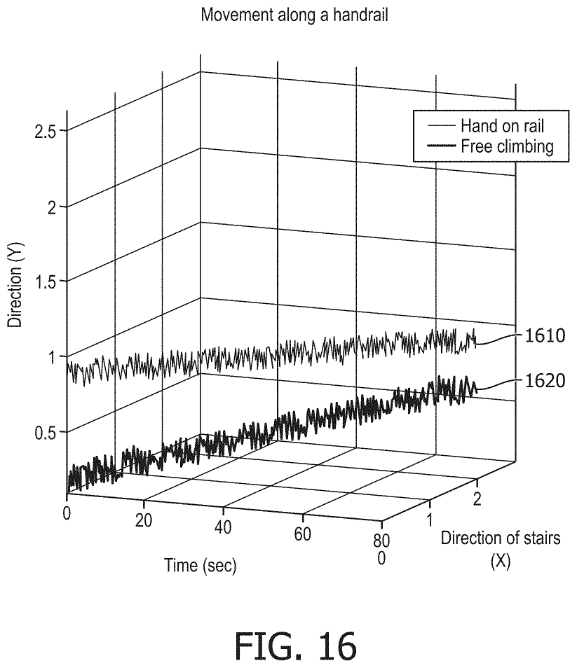

FIG. 16 illustrates another plot generated by the processor based on the data from the wrist-worn sensor. In this case, two curves are shown. The first curve 1610 represents data points corresponding to when the patient was determined to use the handrail on the stairs. The second curve 1620 represents data points corresponding to when the patient was determined to free climb the stairs, e.g., without using the handrail.

From the plot of FIG. 16, use of the handrail is more apparent when compared with the data points generated when the patient walks freely up the stairs. For example, the direction is not simply parallel to the direction of the climb and there is a 40 cm change in the y-coordinate as the patient goes up the stairs, both parameters of which may be identified by the processor performing a comparative analysis of the data. In interpreting the data, the processor may identify that use of the handrail restricts the motion of the person to the vertical and dorsal/ventral directions of the patient. When the patient keeps his hand on the railing, the processor may determine that there is a relatively minimal amount of lateral movement and thus the y-coordinate is unchanged.

From this analysis of the data, the processor may estimate the line of the railing with accurately using the wrist body sensor. In one embodiment, this may be determined, for example, using a line of best fit algorithm, a linear trend estimation algorithm, a residual analysis algorithm, or by using a pre-mapped railing location.

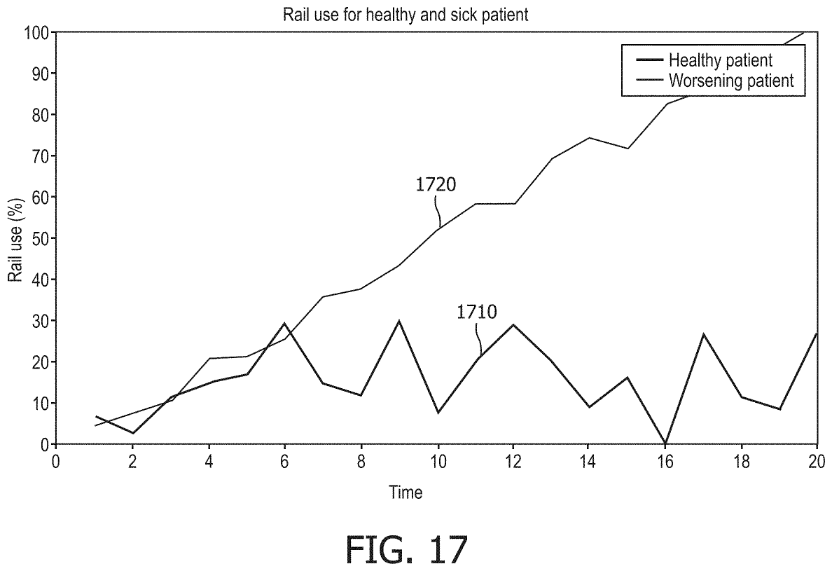

FIG. 17 illustrates an example of a graph which plots handrail usage as a percentage. For example, in this graph, use of the handrail may be expressed as a percentage of time the hand of the patient (wearing the sensor) is on the railing. This percentage time may be calculated by the processor, for example, as: Percentage on railing=(time hand on railing)/(time walking up/down staircase). Similar to other metrics described herein, outlier/anomaly detection may be used by the processor as a basis for identifying when the patient is responding differently. For example, a first curve 1710 generated from the sensor data indicates a percentage of handrail usage by a healthy patient (e.g., one without or only a mild COPD condition) based on the sensor data. Such a patient does not require extensive railing support. A second curve 1720 represents a significant different from the first curve, e.g., the second curve indicated that the percentage use of the handrail increased significantly on a percentage basis over the number of uses. From this curve, the processor may determine that the COPD condition of the patient has gotten worse.

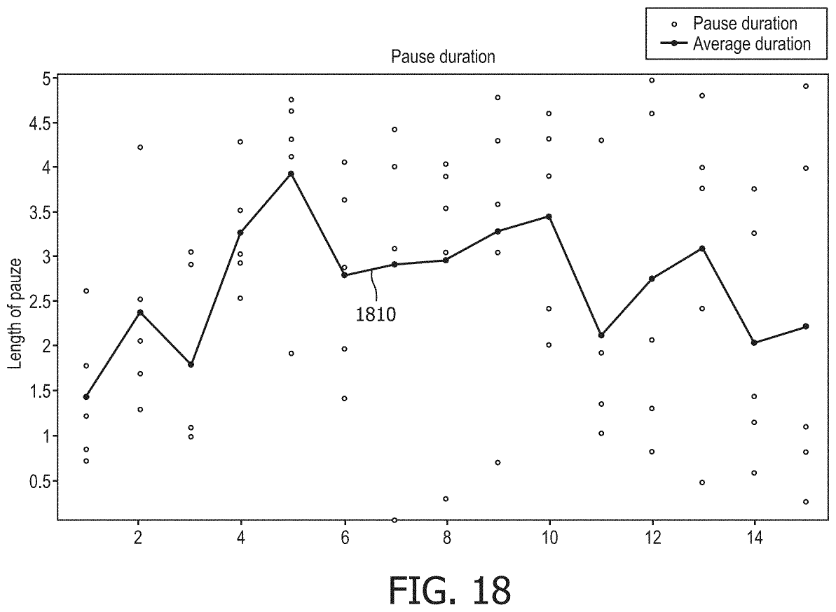

FIG. 18 illustrates an example of a graph that may be generated by the processor of the monitoring system based on the sensor data, this time where the COPD condition of the patient is determined based on the number and/or duration of the times the patient paused when going up or down the steps. The vertical axis of the graph represents pause duration of each episode. The lower horizontal axis represents each stair episode. The upper horizontal axis represents average pause duration over each episode. In FIG. 18, 15 episodes are plotted.

The data points on the graph are generated by the processor (based on the wrist-worn sensor data) and are represented as open circles. The open circles that do not lie on curve 1810 represent pause duration for each stair climbing/descending episode, and the open circles that are connected by curve 1810 represent the average duration of the duration of the pauses. As previously noted, patients who need to stop often may be stopping due to breathlessness. Frequent, lengthy, or multiple pauses during climbing may be used as a basis for indicating a deterioration in the patient condition. By comparing the number of pauses on stairs and the duration of the pauses, the processor may detect a potential change in health. In the 15 stairway climbs plotted in the graph, varying lengths of pauses occurred. The processor may determine the average pause time and use these values to identify outliers. A similar analysis may be performed for the raw number of pauses.

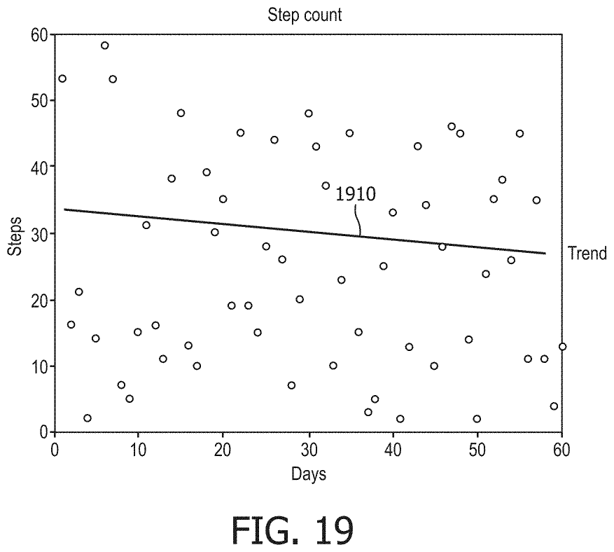

FIG. 19 illustrates an example of a plot including data points indicating the total number of steps on the stairs the patient uses over a time period, e.g., per day. Such a plot may be generated by the processor based on the wrist sensor data, where the vertical axis represents the number of steps/stairs taken per day over a period of days indicated in on the lower horizontal axis. In this case, a 60-day period is taken as an example. The plot may also include step count information (e.g., the number of times the patient ascends ore descends the steps per day) plotted along the upper horizontal axis.

Once generated by the processor, the processor may execute a statistical algorithm to identify patterns that may be used as a basis for determining that a deteriorating lung condition of the patient has discouraged the patient from using steps, or other metrics. From this information, the times of times the patient ascends or descends the steps each day (whether fully or partially) may be determined and this count may be multiplied by the number of steps in the stairwell. If not known, a predetermined standard value such as 10 steps per staircase may be used. Through these techniques, metrics may be generated, for example in the form of trend line 1910, for determining the COPD condition of the patient. For example, a patient who tends to use a lower and lower number of steps per day will be indicated as worse.

The methods, processes, and/or operations described herein may be performed by code or instructions to be executed by a computer, processor, controller, or other signal processing device. The computer, processor, controller, or other signal processing device may be those described herein or one in addition to the elements described herein. Because the algorithms that form the basis of the methods (or operations of the computer, processor, controller, or other signal processing device) are described in detail, the code or instructions for implementing the operations of the method embodiments may transform the computer, processor, controller, or other signal processing device into a special-purpose processor for performing the methods described herein.

Also, another embodiment may include a computer-readable medium, e.g., a non-transitory computer-readable medium, for storing the code or instructions described above. The computer-readable medium may be a volatile or non-volatile memory or other storage device, which may be removably or fixedly coupled to the computer, processor, controller, or other signal processing device which is to execute the code or instructions for performing the operations of the system and method embodiments described herein.

The processors, systems, controllers, and other signal-generating and signal-processing features of the embodiments described herein may be implemented in logic which, for example, may include hardware, software, or both. When implemented at least partially in hardware, the processors, systems, controllers, and other signal-generating and signal-processing features may be, for example, any one of a variety of integrated circuits including but not limited to an application-specific integrated circuit, a field-programmable gate array, a combination of logic gates, a system-on-chip, a microprocessor, or another type of processing or control circuit.

When implemented in at least partially in software, the processors, systems, controllers, and other signal-generating and signal-processing features may include, for example, a memory or other storage device for storing code or instructions to be executed, for example, by a computer, processor, microprocessor, controller, or other signal processing device. The computer, processor, microprocessor, controller, or other signal processing device may be those described herein or one in addition to the elements described herein. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the methods described herein.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method for determining a patient condition, comprising:
receiving sensor information over an observation period;
generating one or more metrics based on the sensor information;
comparing the one or metrics to reference information to determine deviation information; and
generating a risk score indicating a probability that a patient is experiencing an COPD-related event based on the deviation information, wherein the sensor information is received from one or more sensors (30₁ to 30_{N}) at predetermined locations of a patient location.

2. The method of claim 1, wherein the one or more sensors include:
one or more first sensors to track movement of the patient at the patient location throughout the observation period.

3. The method of claim 2, wherein the one or more first sensors track three-dimensional movement of the patient, and the three-dimensional movement includes movement up or down stairs.

4. The method of claim 2, wherein the one or more metrics include a heat map generated based on the information received from the one or more first sensors.

5. The method of claim 4, wherein generating the one or more metrics include generating the heat map to include a first coverage area including one or more routes of movement taken by the patient at the patient location during the observation period.

6. The method of claim 5, wherein comparing the one or more metrics to reference information includes comparing the first coverage area to second coverage area of a heat map corresponding to reference information, the second coverage area corresponding to movement at the patient location for a non-COPD-related event and the deviation information corresponding to a difference between the first coverage area and the second coverage area.

7. A monitoring system, comprising:
an interface to receive sensor (30₁ to 30_{N}) information over an observation period;
a memory storing instructions for determining a health condition of a patient; and
a processor to execute the instructions to perform operations including:
generating one or more metrics based on the sensor information;
comparing the one or metrics to reference information to determine deviation information; and
generating a risk score indicating a probability that a patient is experiencing an COPD-related event based on the deviation information, wherein the sensor information is received from one or more sensors (30₁ to 30_{N}) at predetermined locations of a patient location.

8. The monitoring system of claim 7, wherein the one or more sensors include:
one or more first sensors (30₁ to 30_{N}) to track movement of the patient at the patient location throughout the observation period.

9. The monitoring system of claim 8, wherein the one or more first sensors track three-dimensional movement of the patient, and the three-dimensional movement includes movement of the patient up and down stairs.

10. The monitoring system of claim 8, wherein the one or more metrics include a heat map generated based on the information received from the one or more first sensors.

11. The monitoring system of claim 10, wherein the processor is to generate the one or more metrics by generating the heat map to include a first coverage area including one or more routes of movement taken by the patient at the patient location during the observation period.

12. The monitoring system of claim 11, wherein the processor is to compare the one or more metrics to reference information by comparing the first coverage area to a second coverage area of a heat map corresponding to reference information, the second coverage area corresponding to movement at the patient location for a non-COPD-related event and the deviation information corresponding to a difference between the first coverage area and the second coverage area.

13. A monitoring system, comprising:
interface means for receiving sensor information over an observation period;
a memory storing instructions for determining a health condition of a patient; and
processing means for performing operations including:
generating one or more metrics based on the sensor information;
comparing the one or metrics to reference information to determine deviation information; and
generating a risk score indicating a probability that a patient is experiencing an COPD-related event based on the deviation information, wherein the sensor information is received from one or more sensors (30₁ to 30_{N}) at predetermined locations of a patient location.

14. The monitoring system of claim 13, wherein the one or more sensors include one or more first sensors (30₁ to 30_{N}) to track movement of the patient at the patient location throughout the observation period.

15. The monitoring system of claim 14, wherein
the one or more metrics include a heat map generated based on the information received from the one or more first sensors, and
the processing means performs comparing the one or more metrics to reference information by:
generating a first coverage area of the heat map corresponding to routes taken by the patient at the patient location during the observation period, the coverage area and routes generated based on the information received from the one or more first sensors; and
comparing the first coverage area to a second coverage area in a heat map corresponding to reference routes of movement at the patient location for a non-COPD-related event, wherein the deviation information corresponds to a difference between the first coverage area and the second coverage area.
